(19) United States
(12) Patent Application Publication
(10) Pub. No.: US 2012/0282350 A1
(43) Pub. Date: Nov. 8, 2012

(54) PERFORMANCE ENHANCING COMPOSITIONS AND METHODS OF USE

(75) Inventor: Jan Remmereit, Volda (NO)

(73) Assignee: LIFE SCIENCE NUTRITION AS, Hovdebygda (NO)

(21) Appl. No.: 13/463,964

(22) Filed: May 4, 2012

Related U.S. Application Data
(60) Provisional application No. 61/482,655, filed on May 5, 2011.

(51) Int. Cl. A61K 31/7012; A61K 36/899; A61K 36/48; A61K 35/56; A61K 33/26; A61K 33/30; A61K 33/34; A61K 33/32; A61P 31/00; A61P 21/00; A61P 19/04; A61P 1/00; A61P 37/00; A61P 13/12; A61P 25/22; A61P 25/00; C07H 5/06

(52) U.S. Cl. 424/638; 536/18.7; 424/750; 424/757; 424/520; 424/646; 424/643; 424/639; 514/23

(57) ABSTRACT

The present invention relates to composition containing sialic acids or precursors, and their use to improve performance-related and social parameters in mammals, particularly humans and horses. The daily dosage is preferably selected from the group consisting of 1 microgram to 100 mg/day, 100 microgram to 20 mg/day, and 200 microgram to 10 mg/day.

### CROSS-REFERENCE TO RELATED APPLICATIONS

This application claims the benefit of U.S. Provisional Application Ser. No. 61/482,655, filed May 5, 2011, the entire contents of which are herein incorporated by reference.

### FIELD OF THE INVENTION

The present invention relates to composition containing sialic acids or precursors, and their use to improve performance-related and social parameters in mammals, particularly humans and horses.

### BACKGROUND OF THE INVENTION

The American Horse Council, The Food and Agriculture Organization of the United Nations and the European Union estimate the worldwide population of horses to be in the area of 60-65 million. The current European population is estimated to 7-8 million horses, with approximately 3.9M horses in Great Britain, Germany, and France. The current North America population is estimated to 12-13 million horses, with 9-10 million horses in the United States and 2-3 million horses in Canada. South America is estimated to have 15-16 million horses and the Asia/Pacific rim population is estimated to 22-23 million horses, with China having 12-13 million horses.

Britain's equine Industry has a combined economic impact of seven billion pounds, over half of which derives from horseracing. The equine industry as a whole provides direct and indirect employment to between 220-270,000 people. Horseracing is Britain's second most popular spectator sport, with over six million racing spectators each year. The UK has a world leading equine veterinary profession with 17,260 registered practicing vets. The total spent on equine healthcare per annum is £ 344 million, of which £ 254 million is veterinary fees. The horse industry in the US contributes $ 39 billion in direct economic impact to the US economy. When indirect and induced spending are included, the industry's economic impact reaches $ 102 billion.

As can be seen, substantial financial resources are devoted to ensuring the health and well being of horses. Likewise, the horse racing industry has a substantial economic impact. Accordingly, there is great need for supplements which enhance the performance of horses, as well as which improve the health and well being of horses.

### SUMMARY OF THE INVENTION

The present invention relates to composition containing sialic acids or precursors, and their use to improve performance-related and social parameters in mammals, particularly humans and horses.

In some embodiments, the present invention provides compositions comprising an effective amount of an agent selected from the group consisting of a sialic acid, a sialic precursor and combinations thereof for use in improving a social or performance parameter in a subject. In some embodiments, the composition comprises at least two isolated and purified sialic acids or sialic acid precursors. In some embodiments, the at least two sialic acids or sialic acid precursors have a purity selected from the group consisting of greater than 90%, 95%, 99%, and 99.5% pure. In some embodiments, the at least two sialic acids or sialic acid precursors are selected from the group consisting of n-glycolylneuraminic acid, n-acetylneuraminic acid and N-Acetyl-D-mannosamine. In some embodiments, the sialic acid or sialic acid precursor are derived from natural sources. In some embodiments, the natural source is a non-animal or non-mammalian source. In some embodiments, the sialic acid or sialic acid precursor are chemically synthesized.

In some embodiments, the compositions further comprise at least one composition selected from the group consisting of Avena sativa extract, Fabaceae extract, and Holothuroidea echinodermata extract. In some embodiments, the compositions further comprise one or more mineral supplements selected from the group consisting of Fe, Zn, Cu and Mn.

In some embodiments, the composition is formulated to provide a daily dosage of said agent selected from the group consisting of a sialic acid, a sialic precursor and combinations thereof, selected from the group consisting of 1 microgram to 100 mg/day, 100 microgram to 20 mg/day, and 200 microgram to 10 mg/day. In some embodiments, the composition is formulated to provide a daily dosage of said agent selected from the group consisting of a sialic acid, a sialic precursor and combinations thereof of 0.1 to 10 mg/150 kg subject/day, 0.3 to 5 mg/150 kg subject/day, and 0.5 to 2 mg/150 kg subject/day. In some embodiments, the composition is formulated as a powder, tablet, bolus, liquid or gel. In some embodiments, the composition is formulated for an administration mode selected from the group consisting of enteral administration, parenteral administration, oral administration, subcutaneous administration, intramuscular administration, and intravenous administration.

In some embodiments, the present invention provides a feed or food product, functional food or beverage, nutritional supplement, dietary supplement or oral delivery vehicle comprising the compositions described above.

In some embodiments, the present invention provides for the use of the compositions described above for providing one or more of the following in a subject: reduction in injuries, reduction in infectious disease, increase in lean muscle mass, increase in oxygen uptake, reduction in lactic acid, increase in workload tolerance, improved joint and ligament health, improved behavior, increase in endurance, faster recuperation after workload, faster recuperation from disease, improved pulse parameters, improved gastric health, improved food utilization, improved appearance, improved muscularity, improved coat quality, and improved kidney function.

In some embodiments, the present invention provides for the use of the compositions described above for providing one or more of the following in a subject: improved sociability, reduction of anxious feelings or nervous behavior, reduction of nervous feelings or nervous behavior, increased feeling of calmness, reduction of one or more symptoms associated with social anxiety disorder, reduction of feeling of embarrassment, improved mood, improved social behavior, decreased shyness, improved trainability, improvement in seeking of companionship and reduced anxiety prior to performance or athletic performance.

In some embodiments, the present invention provides methods of improving a performance parameter in a subject comprising: administering to the subject a composition comprising an effective amount of a sialic acid composition or sialic acid precursor composition sufficient to cause a positive effect on said performance parameter. In some embodiments, the performance parameter is selected from the group consisting of reduction in injuries, reduction in infectious disease, increase in lean muscle mass, increase in oxygen uptake, reduction in lactic acid, increase in workload tolerance, improved joint and ligament health, improved behavior, increase in endurance, faster recuperation after workload, faster recuperation from disease, improved pulse parameters, improved gastric health, improved food utilization, improved appearance, improved muscularity, improved coat quality, enhancing the immune system and improved kidney function. In some embodiments, the sialic acid composition is an N- or O-substituted derivative of neuraminic acid. In some embodiments, the N- or O-substituted derivative of neuraminic acid is selected from the group consisting of n-glycolylneuraminic acid and n-acetylneuraminic acid. In some embodiments, the sialic acid precursor is N-Acetyl-D-mannosamine. In some embodiments, the sialic acid composition comprises a combination or two or more sialic acids or sialic acid precursors.

In some embodiments, the sialic acid composition or sialic acid precursor composition is provided in a daily dosage selected from the group consisting of 1 microgram to 100 mg/day, 100 microgram to 20 mg/day, and 200 microgram to 10 mg/day. In some embodiments, the sialic acid composition or sialic acid precursor composition is provided in a daily dosage selected from the group consisting of 0.1 to 10 mg/150 kg subject/day, 0.3 to 5 mg/150 kg subject/day, and 0.5 to 2 mg/150 kg subject/day. In some embodiments, the administering is selected from the group consisting of enteral administration, parenteral administration, oral administration, sublingual administration, subcutaneous administration, intramuscular administration, and intravenous administration.

In some embodiments, the composition further comprises one or more compositions selected from the group consisting of Avena sativa extract, Fabaceae extract, and Holothuroidea echinodermata extract. In some embodiments, the composition further comprises one or more mineral supplements selected from the group consisting of Fe, Zn, Cu and Mn.

In some embodiments, the subject is a mammal In some embodiments, the mammal is selected from the group consisting of a human, horse, bovine, swine and sheep.

In some embodiments, the composition is provided in a formulation selected from the group consisting of powder, liquid and gel. In some embodiments, the composition is provided in a feed or food product, functional food or beverage, nutritional supplement, dietary supplement or oral delivery vehicle. In some embodiments, the sialic acid or sialic acid precursor have a purity selected from the group consisting of greater than 90%, 95%, 99%, and 99.5% pure. In some embodiments, the sialic acid or sialic acid precursor are derived from natural sources. In some embodiments, the natural source is a non-animal or non-mammalian source. In some embodiments, the sialic acid or sialic acid precursor are chemically synthesized.

In some embodiments, the present invention provides methods of improving a social parameter in a subject comprising administering to the subject a composition comprising an effective amount of a sialic acid composition or sialic acid precursor composition sufficient to cause a positive effect on said performance parameter. In some embodiments, the social parameter is selected from the group consisting of improved sociability, reduction of anxious feelings or nervous behavior, reduction of nervous feelings or nervous behavior, increased feeling of calmness, reduction of one or more symptoms associated with social anxiety disorder, reduction of feeling of embarrassment, improved mood, improved social behavior, decreased shyness, improved trainability, improvement in seeking of companionship and reduced anxiety prior to performance or athletic performance.

In some embodiments, the sialic acid composition is an N- or O-substituted derivative of neuraminic acid. In some embodiments, the N- or O-substituted derivative of neuraminic acid is selected from the group consisting of n-glycolylneuraminic acid and n-acetylneuraminic acid. In some embodiments, the sialic acid precursor is N-Acetyl-D-mannosamine. In some embodiments, the sialic acid composition comprises a combination or two or more sialic acids or sialic acid precursors.

In some embodiments, the sialic acid composition or sialic acid precursor composition is provided in a daily dosage selected from the group consisting of 1 microgram to 100 mg/day, 100 microgram to 20 mg/day, and 200 microgram to 10 mg/day. In some embodiments, the sialic acid composition or sialic acid precursor composition is provided in a daily dosage selected from the group consisting of 0.1 to 10 mg/150 kg subject/day, 0.3 to 5 mg/150 kg subject/day, and 0.5 to 2 mg/150 kg subject/day. In some embodiments, the administering is selected from the group consisting of enteral administration, parenteral administration, oral administration, sublingual administration, subcutaneous administration, intramuscular administration, and intravenous administration.

In some embodiments, the composition further comprises one or more compositions selected from the group consisting of Avena sativa extract, Fabaceae extract, and Holothuroidea echinodermata extract. In some embodiments, the composition further comprises one or more mineral supplements selected from the group consisting of Fe, Zn, Cu and Mn.

In some embodiments, the subject is a mammal. In some embodiments, the mammal is selected from the group consisting of a human, horse, bovine, swine and sheep.

In some embodiments, the composition is provided in a formulation selected from the group consisting of powder, liquid and gel. In some embodiments, the composition is provided in a feed or food product, functional food or beverage, nutritional supplement, dietary supplement or oral delivery vehicle. In some embodiments, the sialic acid or sialic acid precursor have a purity selected from the group consisting of greater than 90%, 95%, 99%, and 99.5% pure. In some embodiments, the sialic acid or sialic acid precursor are derived from natural sources. In some embodiments, the natural source is a non-animal or non-mammalian source. In some embodiments, the sialic acid or sialic acid precursor are chemically synthesized.

### DESCRIPTION OF THE FIGURES

FIG. 1: Body mass increases by 3% after 8 months feed on GRPA. (p=0.006)

FIG. 2: Lactic acid decreases by 22% after 8 months GRPA feed (p=0.002)

FIG. 3: GRPA decreases the blood level of lactic acid significantly after hill-test. (12.6% reduction, p=0.0017) (Lactic acid measured with Lactate Pro)

FIG. 4: GRPA reduces the blood level of CK significantly after 8 months (15% reduction, p=0.014).

FIG. 5: GRPA reduces CK value significantly after conducted hill-test (12.6% reduction, p=0.029)

FIG. 6: GRPA reduces the number of joint injections and flu/cold significantly after 8 months (p=0.00006 and p=0.00004).

### DEFINITIONS

As used herein, “sialic acid” is a generic term for the N- or O-substituted derivatives of neuraminic acid, a monosaccharide with a nine-carbon backbone. It is also the name for the most common member of this group, N-acetylneuraminic acid (NeuSAc or NANA). Sialic acids are found widely distributed in animal tissues and to a lesser extent in other species ranging from plants and fungi to yeasts and bacteria, mostly in glycoproteins and gangliosides. The amino group generally bears either an acetyl or glycolyl group but other modifications have been described. The hydroxyl substituents may vary considerably: acetyl, lactyl, methyl, sulfate, and phosphate groups have been found.

As used herein, the term “sialic acid precursor” refers to a compound that is used in the biosynthesis of a sialic acid.

As used herein, the term “extract” refers to a substance made by extracting a part of a raw material.

As used herein, the term “phytonutrient” refers to organic compounds isolated from plants that have a biological effect, and includes, but is not limited to, compounds of the following classes: isoflavonoids, oligomeric proanthcyanidins, indol-3-carbinol, sulforaphone, fibrous ligands, plant phytosterols, ferulic acid, anthocyanocides, triterpenes, omega 3/6 fatty acids, polyacetylene, quinones, terpenes, cathechins, gallates, and quercitin.

As used herein, the term “functional foods” refers to food products that include biologically active nutraceutical agents.

As used herein, the terms “nutraceutical agent,” and related terms, refer to natural, bioactive chemical compounds that have health promoting, disease preventing or medicinal properties. Examples of nutraceutical agents include, but are not limited to, Allium Cepa, Allium Sativum, Aloe Vera, Angelica Species, Naturally Occurring Antioxidants, Aspergillus Oryzae Enzyme Therapy, barley grass, Bromelain, Carnitine, Carotenoids and Flavonoids, Catechin, Centella Asiatica (Gotu kola), Coenzyme Q10, Chinese Prepared Medicines, Coleus Forskohlii, Commiphora Mukul, Crataegus Oxyacantha (Hawthorne), Curcuma Longa (Turmeric), Echinacea Species (Purple Coneflower), Eleutherococcus Senticosus (Siberian Ginseng), Ephedra Species, Dietary Fish Oil Consumption and Fish Oil Supplementation, Genistein, Ginkgo Biloba, Glycyrrhiza (Licorice), Hypericum Perforatum (St. John's Wort), Hydrastis (Goldenseal) and Other Berberine-Containing Plants, Lactobacillus, Lobelia (Indian Tobacco), Melaleuca Alternifolia, Mentha Piperita, NGNA, Panax Ginseng, Pancreatic Enzymes, Piper Mythisticum, Procyanidolic Oligomers, Pygeum Africanum, Quercetin, Sarsaparilla Species, Serenoa Repens (Saw palmetto, Sabal serrulata), Silybum Marianum (Milk Thistle), Rosemary/Lemon balm, Selenite, Tabebuia Avellanedae (LaPacho), Taraxacum Officinale, Tanacetum Parthenium (Feverfew), Taxol, Uva Ursi (Bearberry), Vaccinium Myrtillus (Blueberry), Valerian Officinalis, Viscum Album (Mistletoe), Vitamin A, Beta-Carotene and Other Carotenoids, and Zingiber Officinale (Ginger).

As used herein, the terms “subject” and “patient” refer to any animal, such as a mammal (e.g., human, horse, dog, cat, bovine, swine, and sheep), bird, or fish.

As used herein, the term “physiologically acceptable carrier” refers to any carrier or excipient commonly used with pharmaceuticals. Such carriers or excipients include, but are not limited to, oils, starch, sucrose and lactose.

As used herein, the term “oral delivery vehicle” refers to any means of delivering a pharmaceutical orally, including, but not limited to, capsules, pills, tablets and syrups.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “dietary supplement” refers to a small amount of a compound for supplementation of a human or animal diet packaged in single or multiple does units. Dietary supplements do not generally provide significant amounts of calories but may contain other micronutrients (e.g., vitamins or minerals).

As used herein, the term “nutritional supplement” refers to a composition comprising a “dietary supplement” in combination with a source of calories. In some embodiments, nutritional supplements are meal replacements or supplements (e.g., nutrient or energy bars or nutrient beverages or concentrates).

### DETAILED DESCRIPTION OF THE INVENTION

The present invention relates to composition containing sialic acids or precursors, and their use to improve performance-related parameters in mammals, particularly horses. Accordingly, in some embodiments, the present invention provides compositions comprising an effective amount of a sialic acid or sialic precursor.

The use of a variety of sialic acids is contemplated. Examples include, but are not limited to, N- or O-substituted derivatives of neuraminic acid, and in particularly preferred embodiments are n-glycolylneuraminic acid, n-acetylneuraminic acid. Likewise, the use of a variety of sialic precursors is contemplated, including but not limited to, N-Acetyl-D-mannosamine. In some preferred embodiments, the sialic acid or sialic acid precursor have a purity selected from the group consisting of greater than 90%, 95%, 99%, and 99.5% pure. The use of sialic acids and precursors from a variety sources is contemplated. In some embodiments, the sialic acid or sialic acid precursor are derived from natural sources. In some embodiments, the natural source is a non-animal source. In some embodiments, the sialic acid or sialic acid precursor are chemically synthesized.

In some preferred embodiments, the sialic acid is NGNA. NGNA compositions of the present invention may be delivered in any suitable format. In some embodiments, NGNA is provided from sea cucumbers, e.g., an extract of sea cucumbers, or is prepared from chitin. In some embodiments, the NGNA is preferably about greater than 90%, 95%, 99% or 99.9% pure. In some embodiments, the NGNA is HPLC purified. In some embodiments, NGNA is prepared as described in WO 00/38967, incorporated by reference herein its entirety. For example, N-glycolylneuraminic acid can be purchased commercially from, for example, Sigma Chemical Company, St. Louis, Mo. N-glycolylneuraminic acid also can be synthesized. For example, CMP-N-acetylneuraminic acid hydroxylase can be used to synthesize N-glycolylneurarninic acid as its CMP-glycoside. See, Schlenzka et al., Glycobiolog, 1994, 4(5):675-683. Non-enzymatic methods of synthesis include, for example, synthesis from N-acetylneuraminic acid using methanol or hydrochloric acid and benzylalcohol. Other synthesis methods are described in Choi et al., J. Org. Chem., 1996, 61:8/39 (from mannosamine), Fullard et al., J. Physiol. Chem.′ 1965, 344:167 (from glucosamine), U.S. Pat. No. 4,774,326 and U.S. Pat. No. 4,774,327, both of which are incorporated by reference herein in their entirety.

In some embodiments, compositions comprising a combination of sialic acids and sialic acid precursors are provided. In some embodiments, the compositions comprise at least two isolated and purified sialic acids or sialic acid precursors. In some embodiments, the at least two sialic acids or sialic acid precursors have a purity selected from the group consisting of greater than 90%, 95%, 99%, and 99.5% pure. In some embodiments, the at least two sialic acids or sialic acid precursors are selected from the group consisting of n-glycolylneuraminic acid, n-acetylneuraminic acid and N-Acetyl-D-mannosamine. In some embodiments, the composition may comprise two or more of the following sialic acids: Neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-β-methyl-neuraminic acid, 5-N-Acetyl-8-O-sulpho-neuraminic acid, 5-N-Acetyl-9-β-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-β-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-S—N-glycolyl-8-O-methyl-neuraminic acid, 7,9-di-O-Acetyl-S—N-glycolyl-8-O-methyl-neuraminic acid, 5-N-glycolyl-8-O-sulpho-neuraminic acid, N—(O-acetyl)glycolylneuraminic acid, N—(O-Methyl)glycolylneuraminic acid, 2-Deoxy-2,3-didehydro-5-N-glycolyl-neuraminic acid, 9-O-Acetyl-2-deoxy-2,3-didehydro-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, and 9-O-Acetyl-2-keto-3-deoxynononic acid.

In some embodiments, the first sialic acid or precursor and second sialic acid precursor are provided at a ratio of about 20:1 to 1:20, 10:1 to 1:10, 5:1 to 1:5, 2:1 to 1:2, 20:1 to 10:1, 10:1 to 5:1, 5:1 to 2:1, 2:1 to 1:1. In some embodiments, the first sialic acid or precursor may be one of n-glycolylneuraminic acid, n-acetylneuraminic acid and N-Acetyl-D-mannosamine and the second sialic acid or precursor may be another of n-glycolylneuraminic acid, n-acetylneuraminic acid and N-Acetyl-D-mannosamine. In some embodiments, the composition may comprise three or more sialic acids or precursors.

In some embodiments, the present invention provides compositions or formulations that are formulated to provide a daily dosage of the sialic acid or precursor (or combination thereof) selected from the group consisting of 1 microgram to 100 mg/day, 100 microgram to 20 mg/day, and 200 microgram to 10 mg/day. In some embodiments, the composition is formulated to provide a daily dosage of the sialic acid or precursor of 0.1 to 10 mg/150 kg subject/day, 0.3 to 5 mg/150 kg subject/day, and 0.5 to 2 mg/150 kg subject/day. In some embodiments, an effective amount comprises the daily dosages just described.

In some embodiments, the compositions further comprise one or more extracts. In some embodiments, the extract is selected from the group consisting of an Avena sativa extract, a Fabaceae extract, and a Holothuroidea echinodermata extract and combinations thereof. In some embodiments, the extracts are aqueous extracts, while in other embodiments, the extract is an ethanol extract, a super critical fluid extract, or an organic solvent extract. Oats (Avena sativa) is a species of cereal grain grown for its seed and rich on vitamin B and several minerals. One common use is as livestock feed and oats make up a part of the daily diet of horses, up to about 20 percent of daily intake. Fabaceae or Leguminosae is the third largest family of flowering plants, which is commonly known as the legume family. Grain legumes are cultivated for their seeds, which are used for human and animal consumption. Grain legumes include herbaceous plants like beans, lentils, lupines, peas and peanuts. Holothuroidea echinodermata is the sea cucumber. The compositions of the present invention preferably comprise one or more extracts from these sources.

In some embodiments, the compositions of the present invention further comprise one or more minerals or salts or derivatives thereof, in known effective amounts for daily supplementation for the desired subject. Examples of such minerals include Fe, Zn, Cu and Mn.

In some embodiments, the composition further comprise one or more phytonutrients, nutraceutical agents, or fatty acids. Examples of suitable phytonutrients and nutraceutical agents include, but are not limited to, carotenoids, flavonoids (including isoflavonoids and neo flavonoids; e.g., Luteolin, Apigenin, Tangeritin, Quercetin, Kaempferol, Myricetin, Fisetin, Isorhamnetin, Pachypodol, Rhamnazin, Hesperetin, Naringenin, Eriodictyol, Homoeriodictyol, Taxifolin (or Dihydroquercetin), Dihydrokaempferol, Genistein, Daidzein, Glycitein, Catechins (Catechin (C), Gallocatechin (GC), Catechin 3-gallate (Cg), Gallocatechin 3-gallate (GCg)), Epicatechins (Epicatechin (EC), Epigallocatechin (EGC), Epicatechin 3-gallate (ECg), Epigallocatechin 3-gallate (EGCg)), Cyanidin, Delphinidin, Malvidin, Pelargonidin, Peonidin, Petunidin), resveratrol, Coenzyme Q10, B6/pyridoxamine, n-acetyl cysteine, phosphatidyl serine, Vitamin E, Vitamin B6 (pyridoxine), Gingko biloba extract (preferably at least 20% ginkgolides).

The compositions of the present invention may be provided in a variety of formulations. In preferred embodiments, the formulations provide the preferred daily dosage of the sialic acid or precursor. In some embodiments, the compositions are formulated for an administration mode selected from the group consisting of enteral administration, parenteral administration, oral administration, subcutaneous administration, intramuscular administration, and intravenous administration.

In some embodiments, the present invention provides oral dosage forms comprising sialic acid compositions as described above. The ingredients of the oral dosage form of this invention are preferably contained in acceptable excipients and/or carriers for oral consumption. The actual form of the carrier, and thus, the oral dosage form itself, is not critical. The carrier may be a liquid, gel, gel cap, capsule, powder, solid tablet (coated or non-coated), tea, or the like. The oral dosage form is preferably in the form of a tablet or capsule such as a hard gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.).

In some embodiments, the sialic acid composition is provided in a shot format comprising a daily dosage as described above in an aqueous base of from about 10 to 100 ml, preferably 10 to 80 ml, more preferably about 10 to 60 ml and most preferably about 10 to 50 ml. The shots may preferably comprise one or more of the additional dietary supplements or vitamins listed above and a flavoring agent.

In other embodiments, the compositions are provided as a powder or liquid suitable for adding by the consumer to a feed, food, beverage or water. For example, in some embodiments, the dietary supplement can be administered to a subject in the form of a powder, for instance to be used by mixing into water, or by adding to a feed ration, for example an equine feed ration. In some embodiments, the compositions are provided for use as a top-dressing which can be added to or mixed with a feed ration. In these embodiments, the compositions are formulated to provide the preferred daily dosages of sialic acids or precursors when added to the feed ration. As a non-limiting example, the compositions may be provided as a powder in which ten grams of the powder contains 1 mg of sialic acid or precursor. The powder may then be added to the daily feed ration of horse at a rate of 10 grams per 150 kg of the weight of the horse (i.e., a horse weighing 450 kg would receive 30 grams of powder per day).

The compositions of the present invention may also contain optional ingredients including, for example, herbs, vitamins, minerals, enhancers, colorants, sweeteners, flavorants, inert ingredients, and the like. In some particularly preferred embodiments, the ingredient is suitable for feeding to a horse, for example, molasses, apple flavoring, cane sugar, and the like.

In some embodiments, the compositions further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide.

Many different feed rations may be formulated for animals from many different feed ingredients. Rations are generally formulated to provide-nutrients in accordance with National Research Council standards. The feedstuffs used in the ration are chosen according to market price and availability. Thus, some components of the ration may change over time. In the feeds of the present invention, the ration will preferably contain compositions described above in an amount sufficient to deliver the preferred daily dosage, but other components may vary over time based on the price of the component. For discussions on feed ration formulation, actual rations and NRC guidelines, see Church, Livestock Feeds and Feeding, O&B Books, Inc., Corvallis Oreg. (1984) and Feeds and Nutrition Digest, Ensminger, Oldfield and Heineman eds., Ensminger Publishing Corporation, Clovis, Calif. (1990), incorporated herein by reference.

The animal feed rations of the present invention may be characterized according to NRC requirements. NRC requirements may be found in Church, Livestock Feeds and Feeding, O&B Books, Inc., Corvallis Oreg. (1984), or other nutritional standards. Equine and other animal rations are traditionally balanced using the protein and energy requirements, and then adjusted if needed to meet the other requirements. The equine and other feeds of the present invention will contain sialic acids or precursors in amounts sufficient to deliver the preferred daily dosages, plus other feed materials necessary to balance the feed to meet the NRC requirements for the different stages of growth and maintenance.

The relative amounts of protein and energy are preferably adjusted to reflect Nutritional Standards requirements or requirement of a trainer, for example a trainer or race or working horses. The amounts of feed components will vary with the stage of animal fed. A growing ration for young animals will have higher protein levels, while a finishing ration for finishing animals for market will have higher energy values which are supplied by carbohydrates.

In a preferred embodiment, the compositions described above are incorporated into a pelleted or textured feed for administration to domestic animals. Pelleted feed is created by first mixing feed components and then compacting and extruding the feed components through a die with heat and pressure. The feed is pelleted by methods known in the art, which are described in MacBain, Pelleting Animal Feed, American Feed Manufacturers Association, Arlington, Va. (1974), incorporated herein by reference.

Rations containing the foregoing compositions may also be formulated for animals other than horses. The amount of CFAP administered is not critical as long as it is enough to be effective in decreasing body fat and increasing feed efficiency or other benefits. The feeds are formulated as above, and tailored to the requirements of the animal to be fed in accordance with NRC guidelines. For example, feeds may be formulated for dogs, cats, poultry, sheep and cattle. Various rations are given in the examples. Various feed formulations, balancing methods and requirements for these animals are discussed in Church, Livestock Feeds and Feeding, O&B Books, Inc., Corvallis Oreg. (1984) and Feeds and Nutrition Digest, Ensminger, Oldfield and Heineman eds., Ensminger Publishing Corporation, Clovis, Calif. (1990), incorporated herein by reference. It should be borne in mind that feed companies and livestock producers develop their own nutritional requirements based on historical results. The present invention makes the use of CFAP possible in these various feeds because the CFAP may be produced economically in bulk quantities.

In other embodiments, the present invention provides nutritional supplements (e.g., energy bars or meal replacement bars or beverages) comprising the foregoing compositions. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutritional supplement can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

The nutritional supplement can be provided in a variety of forms, and by a variety of production methods. In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

Servings of the nutritional supplement preferably contain between 0.1 g and 10.0 g of NGNA, preferably between 0.5 and 2.0 g NGNA, and even more preferably approximately 1.0 g NGNA. It is understood by those of skill in the art that other ingredients can be added to those described herein, for example, fillers, emulsifiers, preservatives, etc. for the processing or manufacture of a nutritional supplement.

In still further embodiments, the present invention provides functional foods, including food products, prepared food products, or foodstuffs comprising nutraceutical agents. For example, in some embodiments, beverages and solid or semi-solid foods comprising nutraceutical agents are provided. These forms can include, but are not limited to, beverages (e.g., water, soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

The compositions described above are useful for improving performance parameters as well as increasing overall health in animals, including mammals (e.g., human, horse, dog, cat, bovine, swine, and sheep), birds, and fish (i.e., subjects). In some embodiments, the compositions are provided to a subject in amount sufficient to cause one or more of the following effects: reduction in injuries, reduction in infectious disease, increase in lean muscle mass, increase in oxygen uptake, reduction in lactic acid, increase in workload tolerance, improved joint and ligament health, improved behavior, increase in endurance, faster recuperation after workload, faster recuperation from disease, improved pulse parameters, improved gastric health, improved food utilization, improved appearance, improved muscularity, improved coat quality, and improved kidney function. In some embodiments, the compositions are included in a diet or used to supplement a subject to ameliorate the side effects or toxic effects of an agent that is being supplied to the subject, for example, an agent that reduces one or more of the performance factors, that inhibits the immune system (e.g., a steroid), or that has an adverse or toxic effect on the kidneys.

The composition of the present invention are also useful in improving social parameters in animals, including mammals (e.g., human, horse, dog, cat, bovine, swine, and sheep), birds, and fish (i.e., subjects). Interestingly, it have been found that the compositions of the present invention, especially sialic acid compositions, improve social parameters in animals, including mammals (e.g., human, horse, dog, cat, bovine, swine, and sheep), birds, and fish. In some embodiments, the social parameter is improved sociability, reduction of anxious feelings or nervous behavior, reduction of nervous feelings or nervous behavior, increased feeling of calmness, reduction of one or more symptoms associated with social anxiety disorder (e.g., fear of social situations, nervousness, avoidance of social situations, confusion, pounding heart, sweating, shaking, blushing, muscle tension, upset stomach, and diarrhea), reduction of feeling of embarrassment, improved mood, improved social behavior, decreased shyness, improved trainability, improvement in seeking of companionship, decreased anxiety prior to athletic performance.

### EXAMPLES

### Example 1

### Formulation of GRPA

| | Specification | Result |
|---|---|---|
| Appearance | Light yellow powder | Conform |
| Loss on drying | 10% max. | 6.45% |
| GRPA | 78-82% | 80% |
| Fe | 2.2-2.6% | 2.42% |
| Zn | 1.6-1.9% | 1.76% |
| Cu | 1.1-1.4% | 1.26% |
| Mn | 0.6-0.7% | 0.65% |

GRPA is:
NGNA: 1 mg/10 g GRPA w/w
Avena sativa extract powder: QS
Fabaceae extract powder: QS
Holothuroidea echinodermata extract powder: QS
Metabolized Energy: >Kcal 500/kg.
Yeast and Mold: negative
E. Coli: Negative
Arsenic: 3 ppm max.
Heavy Metal: 20 ppm max

### Example 2

### Effects of Feeding GRPA

GRPA Increases Muscle Mass.

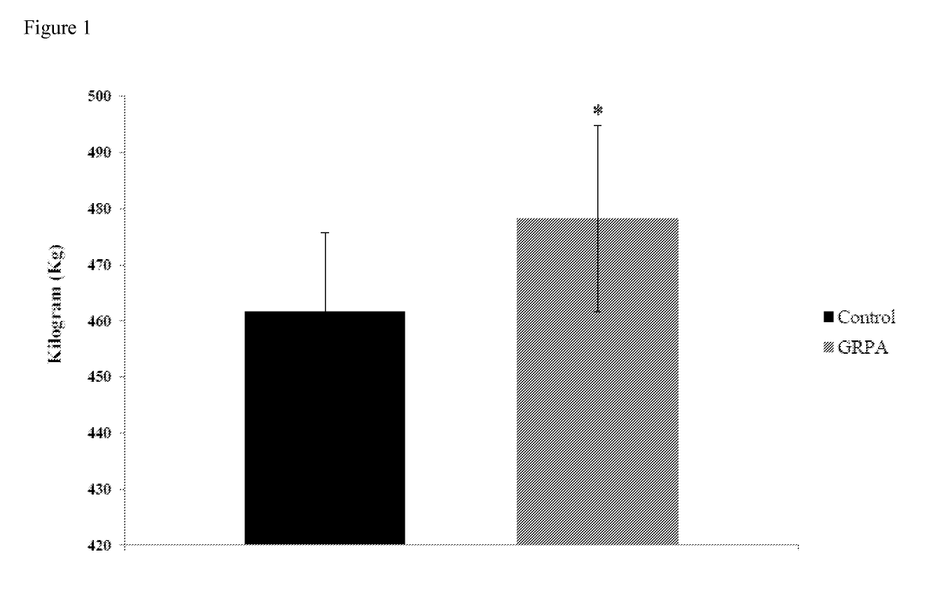

Increase in muscle mass is an enormous advantage for the performance level during horse race competition, increasing the ability of the horse's efficiency and speed. Upon regular training GRPA increases body weight, thus muscle mass. For results see FIG. 1.

GRPA Increases Muscular Endurance.

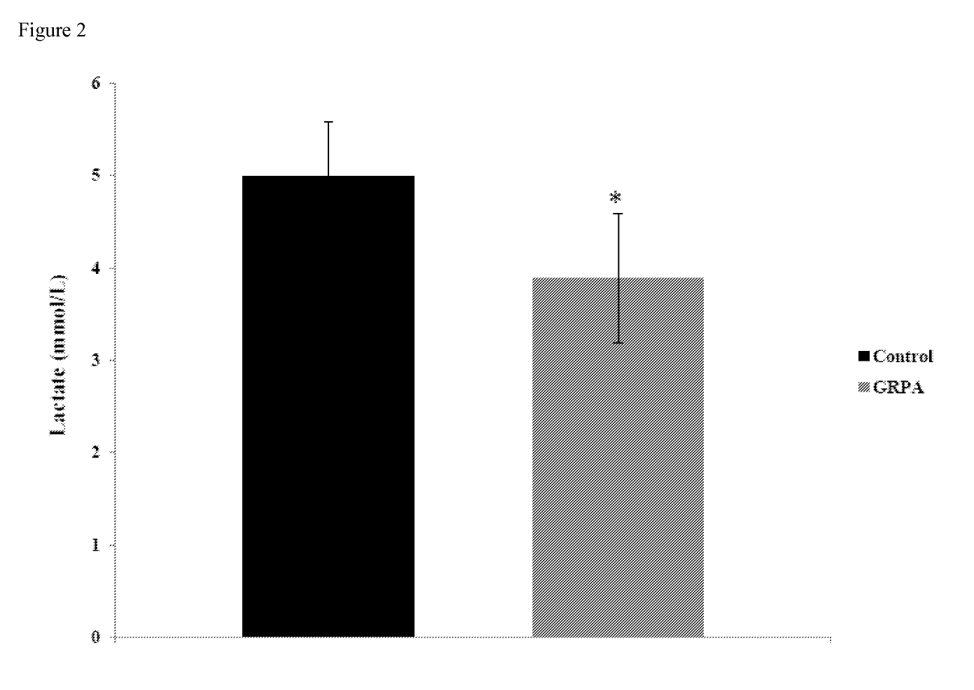

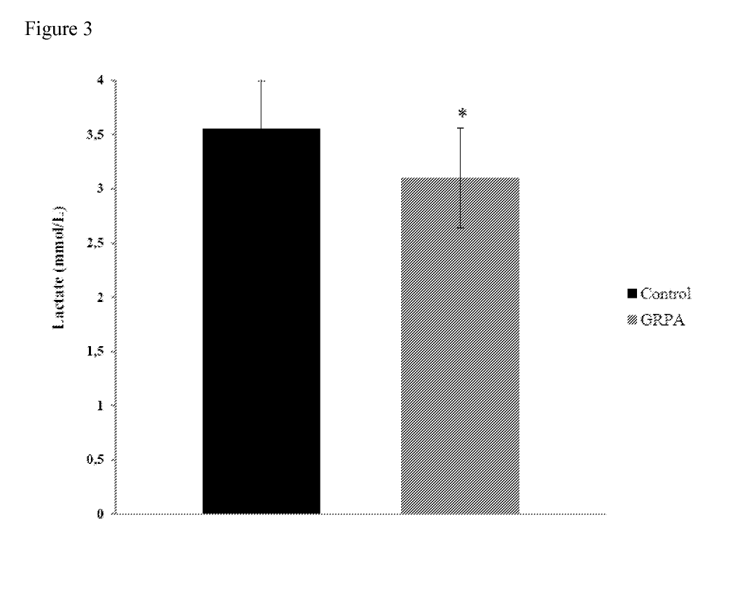

With racing and heavy training the blood level of lactic acid increases, however, the level of lactic acid production depends on the horse's ability for oxygen consumption and oxygen delivery to the muscles. With high levels of lactic acid the muscle tires and decreases horses ability to keep high speed under racing. GRPA increases muscular endurance by significantly reducing the blood level of lactic acid. This gives the race horse a great advantage. Horses who feed on GRPA will have improved muscular endurance and the ability to keep high speed under racing, without tiring muscles. See FIG. 2: Lactic acid decreases by 22% after 8 months GRPA feed (p=0.002), and FIG. 3: GRPA decreases the blood level of lactic acid significantly after hill-test. (12, 6% reduction, p=0.0017) (Lactic acid measured with Lactate Pro)

GRPA Increases Workload Tolerance.

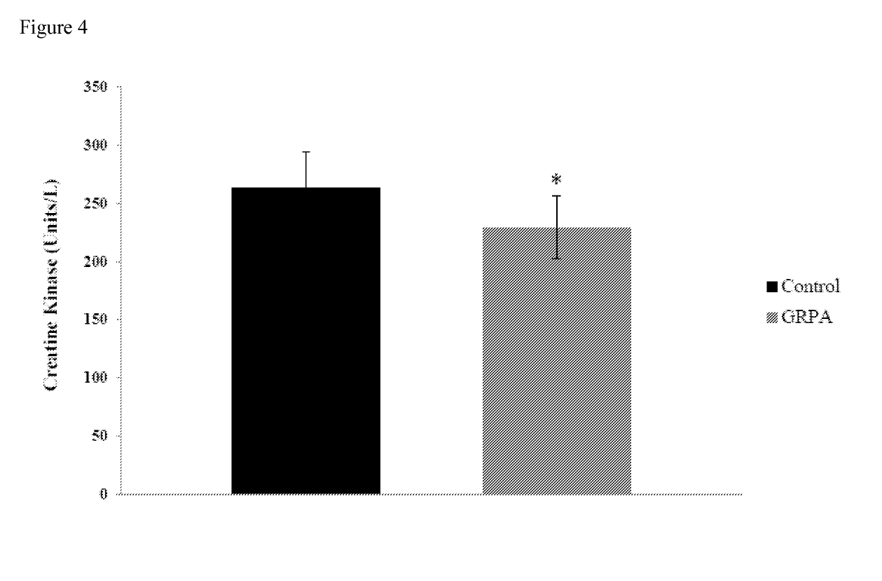

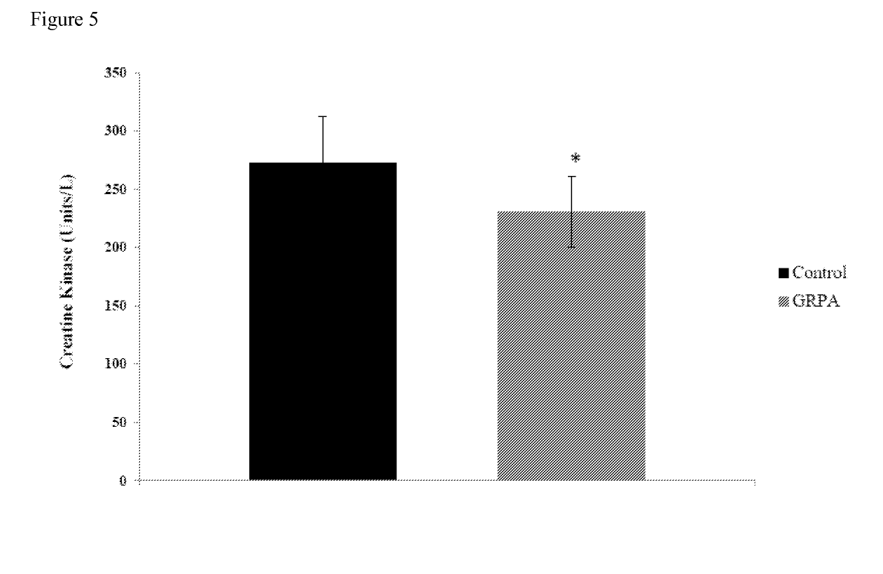

After workload the horse and the muscles need time to recover. Recovery time needed depends on e.g. performed workload and the horse's ability to recover. Workload tolerance is monitored by measuring blood level of CK. Increased CK values indicates muscle breakdown, and the horse needs a longer recovery period. With GRPA, CK value is decreased with 12.6% compared to control, resulting in reduced recovery time and increased workload tolerance, as well as increased ability to hard training without incidents of injuries. See FIG. 4: GRPA reduces the blood level of CK significantly after 8 months (15% reduction, p=0.014), and FIG. 5: GRPA reduces CK value significantly after conducted hill-test (12.6% reduction, p=0.029)

GRPA Increases the Immune System.

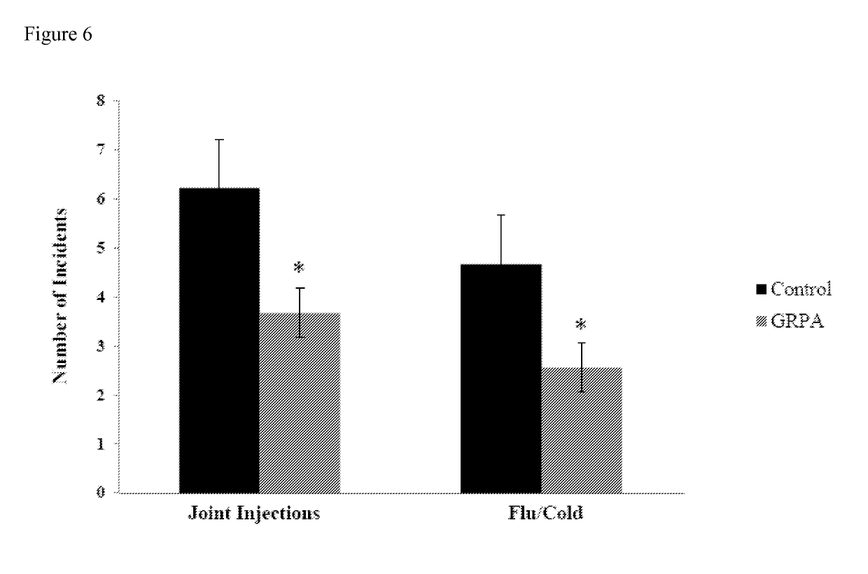

Race horses are exposed to systematic hard workload and this increases the chance of injuries as well as incidents of general infections. The advantage of GRPA feed is that it reduces incidents of injuries with 41% and cold/flu with 45% compared to control group. See FIG. 6: GRPA reduces the number of joint injections and flu/cold significantly after 8 months (p=0.00006 and p=0.00004)

Concluding Remarks:

GRPA gives a uniquely advantageous effect as a feed for race horses, by improving the horse's performance, growth and health, with no side effects.

### Example 3

### Social Effects of Sialic Acid Supplements

A sialic acid supplement (GRPA) was provided to horses. Trainers noted improvement of social parameters in the animals fed the supplement, including generally improved sociability, improved trainability, reduced aggression, and increased contact-seeking behavior.

### Example 4

### Effect of Sialic Administration in a Human Subject

A sialic supplement comprising 10 mg NANA and NGNA was consumed by a small group of human fitness enthusiasts. The preferred administration was route was either nasal or sublingual administration, which showed the most immediate effect. Oral administration was also effective, although the effects were less immediate. The subjects noted the following effects during the trial: reduced susceptibility to colds and flu, improved energy, improved mood and sociability, improved social interaction and seeking social interaction, improved feeling of well being, improved lean body mass, decreased anxiety prior to athletic performance, and improved training intensity and results.

### Example 5

### Formulation of a Sialic Acid Supplement

A sialic acid supplement is formulated by combining 10 mg of n-glycolylneuraminic acid and 10 mg n-acetylneuraminic acid, each greater than 95% pure with 480 mg maltodextrin and encapsulating the mixture in a gel capsule.

### Example 6

### Formulation of a Sialic Acid Supplement

A sialic acid supplement is formulated by combining 100 mg of n-glycolylneuraminic acid and 100 mg n-acetylneuraminic acid, each greater than 95% pure with 300 mg maltodextrin and encapsulating the mixture in a gel capsule.

### Example 7

### Formulation of a Sialic Acid Supplement

A sialic acid supplement is formulated by combining 10 mg of n-glycolylneuraminic acid and 10 mg N-Acetyl-D-mannosamine, each greater than 95% pure with 480 mg maltodextrin and encapsulating the mixture in a gel capsule.

### Example 8

### Formulation of a Sialic Acid Supplement

A sialic acid supplement is formulated by combining 100 mg of n-glycolylneuraminic acid and 100 mg N-Acetyl-D-mannosamine, each greater than 95% pure with 300 mg maltodextrin and encapsulating the mixture in a gel capsule.

### Example 9

### Formulation of a Sialic Acid Supplement

A sialic acid supplement is formulated by combining 10 mg of n-acetylneuraminic acid and 10 mg N-Acetyl-D-mannosamine, each greater than 95% pure with 480 mg maltodextrin and encapsulating the mixture in a gel capsule.

### Example 10

### Formulation of a Sialic Acid Supplement

A sialic acid supplement is formulated by combining 100 mg of n-acetylneuraminic acid and 100 mg N-Acetyl-D-mannosamine, each greater than 95% pure with 300 mg maltodextrin and encapsulating the mixture in a gel capsule.

### Example 11

### Formulation of a Beverage Containing Sialic Acid

A beverage is formulated by dissolving 50 g of n-glycolylneuraminic acid and 50 g n-acetylneuraminic acid in 100 liters of water. A flavoring agent is added and the mixture is dispensed into 20 ml containers.

### Example 12

### Formulation of a Beverage Containing Sialic Acid

A beverage is formulated by dissolving 50 g of n-glycolylneuraminic acid and 50 g N-Acetyl-D-mannosamine in 100 liters of water. A flavoring agent is added and the mixture is dispensed into 20 ml containers.

### Example 13

### Formulation of a Beverage Containing Sialic Acid

A beverage is formulated by dissolving 50 g of n-acetylneuraminic acid and 50 g N-Acetyl-D-mannosamine in 100 liters of water. A flavoring agent is added and the mixture is dispensed into 20 ml containers.

### Example 11

### Formulation of a Beverage Containing Sialic Acid

A beverage is formulated by dissolving 5 g of n-glycolylneuraminic acid and 5 g n-acetylneuraminic acid in 100 liters of water. The mixture is dispensed into 500 ml containers.

### Example 12

### Formulation of a Beverage Containing Sialic Acid

A beverage is formulated by dissolving 5 g of n-glycolylneuraminic acid and 5 g N-Acetyl-D-mannosamine in 100 liters of water. The mixture is dispensed into 500 ml containers.

### Example 13

### Formulation of a Beverage Containing Sialic Acid

A beverage is formulated by dissolving 5 g of n-acetylneuraminic acid and 5 g N-Acetyl-D-mannosamine in 100 liters of water. The mixture is dispensed into 500 ml containers.

### Example 14

### Use of Sialic Acid Compositions to Improve Performance Parameters

Forty physically active adult volunteers are divided into two groups of 20. Group A receives the sialic acid formulation of Example 5 and Group B receives a placebo. The trial is conducted for a period of 12 weeks. The participants maintain existing workout and exercise patterns. At the end of the trial, Group A reports improvements, as compared to control Group B, in combinations of the following parameters: reduction in injuries, reduction in infectious disease, increase in lean muscle mass, increase in oxygen uptake, reduction in lactic acid, increase in workload tolerance, improved joint and ligament health, increase in endurance, faster recuperation after exercise, faster recuperation from disease, improved pulse parameters, improved gastric health, improved food utilization, improved appearance, improved muscularity, and improved kidney function.

### Example 15

### Use of Sialic Acid Compositions to Improve Performance Parameters

Forty physically active adult volunteers are divided into two groups of 20. Group A receives the sialic acid formulation of Example 7 and Group B receives a placebo. The trial is conducted for a period of 12 weeks. The participants maintain existing workout and exercise patterns. At the end of the trial, Group A reports improvements, as compared to control Group B, in combinations of the following parameters: reduction in injuries, reduction in infectious disease, increase in lean muscle mass, increase in oxygen uptake, reduction in lactic acid, increase in workload tolerance, improved joint and ligament health, increase in endurance, faster recuperation after exercise, faster recuperation from disease, improved pulse parameters, improved gastric health, improved food utilization, improved appearance, improved muscularity, and improved kidney function.

### Example 16

### Use of Sialic Acid Compositions to Improve Social Parameters

Forty adult volunteers are divided into two groups of 20. Group A receives the sialic acid formulation of Example 5 and Group B receives a placebo. The trial is conducted for a period of 12 weeks. At the end of the trial, Group A reports improvements, as compared to control Group B, in combinations of the following parameters: improved sociability, reduction of anxious feelings or nervous behavior, reduction of nervous feelings or nervous behavior, increased feeling of calmness, reduction of one or more symptoms associated with social anxiety disorder (e.g., fear of social situations, nervousness, avoidance of social situations, confusion, pounding heart, sweating, shaking, blushing, muscle tension, upset stomach, and diarrhea), reduction of feeling of embarrassment, improved mood, improved social behavior, decreased shyness, improved trainability, improvement in seeking of companionship.

### Example 17

### Use of Sialic Acid Compositions to Improve Social Parameters

Forty adult volunteers are divided into two groups of 20. Group A receives the sialic acid formulation of Example 7 and Group B receives a placebo. The trial is conducted for a period of 12 weeks. At the end of the trial, Group A reports improvements, as compared to control Group B, in combinations of the following parameters: improved sociability, reduction of anxious feelings or nervous behavior, reduction of nervous feelings or nervous behavior, increased feeling of calmness, reduction of one or more symptoms associated with social anxiety disorder (e.g., fear of social situations, nervousness, avoidance of social situations, confusion, pounding heart, sweating, shaking, blushing, muscle tension, upset stomach, and diarrhea), reduction of feeling of embarrassment, improved mood, improved social behavior, decreased shyness, improved trainability, improvement in seeking of companionship.

We claim:

1. A composition comprising an effective amount of an agent selected from the group consisting of a sialic acid, a sialic precursor and combinations thereof for use in improving a social or performance parameter in a subject.

2. The composition of claim 1, wherein said composition comprises at least two isolated and purified sialic acids or sialic acid precursors.

3. The composition of claim 2, wherein said at least two sialic acids or sialic acid precursors have a purity selected from the group consisting of greater than 90%, 95%, 99%, and 99.5% pure.

4. The composition of claim 2, wherein said at least two sialic acids or sialic acid precursors are selected from the group consisting of n-glycolylneuraminic acid, n-acetylneuraminic acid and N-Acetyl-D-mannosamine.

5. The composition of claim 1, wherein said sialic acid or sialic acid precursor are derived from natural sources.

6. The composition of claim 5, wherein said natural source is a non-animal or non-mammalian source.

7. The composition of claim 1, wherein said sialic acid or sialic acid precursor are chemically synthesized.

8. The composition of claim 1, further comprising at least one composition selected from the group consisting of Avena sativa extract, Fabaceae extract, and Holothuroidea echinodermata extract.

9. The composition of claim 8, further comprising one or more mineral supplements selected from the group consisting of Fe, Zn, Cu and Mn.

10. The composition of claim 1, wherein said composition is formulated to provide a daily dosage of said agent selected from the group consisting of a sialic acid, a sialic precursor and combinations thereof, selected from the group consisting of 1 microgram to 100 mg/day, 100 microgram to 20 mg/day, and 200 microgram to 10 mg/day.

11. The composition of claim 1, wherein said composition is formulated to provide a daily dosage of said agent selected from the group consisting of a sialic acid, a sialic precursor and combinations thereof of 0.1 to 10 mg/150 kg subject/day, 0.3 to 5 mg/150 kg subject/day, and 0.5 to 2 mg/150 kg subject/day.

12. The composition of claim 1, wherein said composition is formulated as a powder, tablet, bolus, liquid or gel.

13. The composition of claim 1, wherein said composition is formulated for an administration mode selected from the group consisting of enteral administration, parenteral administration, oral administration, subcutaneous administration, intramuscular administration, and intravenous administration.

14. A composition comprising a feed or food product comprising the composition of claim 1.

15. A method of improving a performance parameter in a subject comprising:
administering to the subject a composition comprising an effective amount of a sialic acid composition or sialic acid precursor composition sufficient to cause a positive effect on said performance parameter.

16. The method of claim 15, wherein said performance parameter is selected from the group consisting of reduction in injuries, reduction in infectious disease, increase in lean muscle mass, increase in oxygen uptake, reduction in lactic acid, increase in workload tolerance, improved joint and ligament health, improved behavior, increase in endurance, faster recuperation after workload, faster recuperation from disease, improved pulse parameters, improved gastric health, improved food utilization, improved appearance, improved muscularity, improved coat quality, enhancing the immune system and improved kidney function.

17. The method of claim 15, wherein said sialic acid composition is an N- or O-substituted derivative of neuraminic acid.

18. The method of claim 15, wherein said N- or O-substituted derivative of neuraminic acid is selected from the group consisting of n-glycolylneuraminic acid and n-acetylneuraminic acid.

19. The method of claim 15, wherein said sialic acid precursor is N-Acetyl-D-mannosamine.

20. The method of claim 15, wherein said sialic acid composition comprises a combination or two or more sialic acids or sialic acid precursors.

21. The method of claim 15, wherein said sialic acid composition or sialic acid precursor composition is provided in a daily dosage selected from the group consisting of 1 microgram to 100 mg/day, 100 microgram to 20 mg/day, and 200 microgram to 10 mg/day.

22. The method of claim 15, wherein said sialic acid composition or sialic acid precursor composition is provided in a daily dosage selected from the group consisting of 0.1 to 10 mg/150 kg subject/day, 0.3 to 5 mg/150 kg subject/day, and 0.5 to 2 mg/150 kg subject/day.

23. The method of claim 15, wherein said administering is selected from the group consisting of enteral administration, parenteral administration, oral administration, subcutaneous administration, intramuscular administration, and intravenous administration.

24. The method of claim 15, wherein said composition further comprises one or more compositions selected from the group consisting of Avena sativa extract, Fabaceae extract, and Holothuroidea echinodermata extract.

25. The method of claim 15, said composition further comprises one or more mineral supplements selected from the group consisting of Fe, Zn, Cu and Mn.

26. The method of claim 15, wherein said subject is a mammal.

27. The method of claim 26, wherein said mammal is selected from the group consisting of a human, horse, bovine, swine and sheep.

28. The method of claim 15, wherein said composition is provided in a formulation selected from the group consisting of powder, liquid and gel.

29. The method of claim 15, wherein said composition is provided in a food or feed product.

30. The method of claim 15, wherein said sialic acid or sialic acid precursor have a purity selected from the group consisting of greater than 90%, 95%, 99%, and 99.5% pure.

31. The method of claim 15, wherein said sialic acid or sialic acid precursor are derived from natural sources.

32. The method of claim 30, wherein said natural source is a non-animal or non-mammalian source.

33. The method of claim 15, wherein said sialic acid or sialic acid precursor are chemically synthesized.

34. A method of improving a social parameter in a subject comprising:
administering to the subject a composition comprising an effective amount of a sialic acid composition or sialic acid precursor composition sufficient to cause a positive effect on said performance parameter.

35. The method of claim 34, wherein said social parameter is selected from the group consisting of improved sociability, reduction of anxious feelings or nervous behavior, reduction of nervous feelings or nervous behavior, increased feeling of calmness, reduction of one or more symptoms associated with social anxiety disorder, reduction of feeling of embarrassment, improved mood, improved social behavior, decreased shyness, improved trainability, improvement in seeking of companionship.

36. The method of claim 34, wherein said sialic acid composition is an N- or O-substituted derivative of neuraminic acid.

37. The method of claim 34, wherein said N- or O-substituted derivative of neuraminic acid is selected from the group consisting of n-glycolylneuraminic acid and n-acetylneuraminic acid.

38. The method of claim 34, wherein said sialic acid precursor is N-Acetyl-D-mannosamine.

39. The method of claim 34, wherein said sialic acid composition comprises a combination or two or more sialic acids or sialic acid precursors.

40. The method of claim 34, wherein said sialic acid composition or sialic acid precursor composition is provided in a daily dosage selected from the group consisting of 1 microgram to 100 mg/day, 100 microgram to 20 mg/day, and 200 microgram to 10 mg/day.

41. The method of claim 34, wherein said sialic acid composition or sialic acid precursor composition is provided in a daily dosage selected from the group consisting of 0.1 to 10 mg/150 kg subject/day, 0.3 to 5 mg/150 kg subject/day, and 0.5 to 2 mg/150 kg subject/day.

42. The method of claim 34, wherein said administering is selected from the group consisting of enteral administration, parenteral administration, oral administration, subcutaneous administration, intramuscular administration, and intravenous administration.

43. The method of claim 34, wherein said composition further comprises one or more compositions selected from the group consisting of Avena sativa extract, Fabaceae extract, and Holothuroidea echinodermata extract.

44. The method of claim 34, said composition further comprises one or more mineral supplements selected from the group consisting of Fe, Zn, Cu and Mn.

45. The method of claim 34, wherein said subject is a mammal.

46. The method of claim 45, wherein said mammal is selected from the group consisting of a human, horse, bovine, swine and sheep.

47. The method of claim 34, wherein said composition is provided in a formulation selected from the group consisting of powder, liquid and gel.

48. The method of claim 34, wherein said composition is provided in a food or feed product.

49. The method of claim 34, wherein said sialic acid or sialic acid precursor have a purity selected from the group consisting of greater than 90%, 95%, 99%, and 99.5% pure.

50. The method of claim 34, wherein said sialic acid or sialic acid precursor are derived from natural sources.

51. The method of claim 34, wherein said natural source is a non-animal or non-mammalian source.

52. The method of claim 34, wherein said sialic acid or sialic acid precursor are chemically synthesized.
